(19) United States
(12) Patent Application Publication
(10) Pub. No.: US 2012/0035464 A1
(43) Pub. Date: Feb. 9, 2012

(54) CONTROL APPARATUS FOR CONTROLLING A THERAPEUTIC APPARATUS

(75) Inventors: Balasundara Raju, Tarrytown, NY (US); Shunmugavelu Sokka, Brighton, MA (US)

(73) Assignee: KONINKLIJKE PHILIPS ELECTRONICS N.V., EINDHOVEN (NL)

(21) Appl. No.: 13/265,142

(22) PCT Filed: Apr. 13, 2010
(86) PCT No.: PCT/IB10/51592

§ 371 Date: Oct. 19, 2011

Related U.S. Application Data
(60) Provisional application No. 61/170,701, filed on Apr. 20, 2009.

(51) Int. Cl. A61B 5/055; A61B 8/00

(52) U.S. Cl. 600/411

(57) ABSTRACT

A control apparatus (106) for controlling a therapeutic apparatus (100), wherein the control apparatus comprises: —an ultrasound control interface (110) for controlling a therapeutic ultrasound system (102), —a magnetic resonance control interface (112) for controlling a magnetic resonance apparatus (104) adapted for acquiring magnetic resonance imaging data from a subject and for acquiring magnetic resonance spectroscopy data from a subject (244), —an image processing module (124, 126, 128) for generating at least one magnetic resonance imaging image (500) from the magnetic resonance imaging data and for generating at least one magnetic resonance spectroscopy map (502, 514, 516, 518, 520) from the magnetic resonance spectroscopy data, —a planning module (120) adapted for receiving the magnetic resonance imaging image and the magnetic resonance spectroscopy map and for outputting planning data (732), —a control module (122) adapted for controlling the therapeutic ultrasound system using the ultrasound control apparatus using the planning data, wherein the control module is further adapted for controlling the acquisition of the acquiring magnetic resonance imaging data and magnetic resonance spectroscopy data using the magnetic resonance control interface.

### FIELD OF THE INVENTION

The invention relates to the control of therapeutic ultrasonic apparatuses, in particular to the control of ultrasonic apparatuses that plan therapy using magnetic resonance imaging and magnetic resonance spectroscopy.

### BACKGROUND OF THE INVENTION

Ultrasound from a focused ultrasonic transducer can be used to selectively treat regions within the interior of the body. Ultrasonic waves are transmitted as high energy mechanical vibrations. These vibrations induce tissue heating as they are damped, and they can also lead to cavitation. Both tissue heating and cavitation can be used to destroy tissue in a clinical setting. However, heating tissue with ultrasound is easier to control than cavitation. Ultrasonic treatments can be used to ablate tissue and to kill regions of cancer cells selectively. This technique has been applied to the treatment of uterine fibroids, and has reduced the need for hysterectomy procedures. At lower powers or in pulsed mode, ultrasound can be used to selectively deliver genetic material or medicine to a region.

To perform ultrasonic therapy, a focused ultrasonic transducer can be used to focus the ultrasound on a particular treatment volume. The transducer is typically mounted within a medium, such as degassed water, that is able to transmit ultrasound. Actuators are then used to adjust the position of the ultrasonic transducer and thereby adjust the tissue region that is being treated.

Magnetic Resonance Imaging (MRI) can be used to plan the ultrasound treatment and can also be used to guide the treatment. U.S. Pat. No. 7,343,030 B2 discloses a system and method for analyzing images of tumors for the purpose of diagnosis and planning.

A static magnetic field is used by MRI scanners to align the nuclear spins of atoms as part of the procedure for producing images within the body of a patient. This static magnetic field is referred to as the polarizing or B0 field.

During an MRI scan, Radio Frequency (RF) pulses generated by a transmitter coil cause perturbations to the local magnetic field, and RF signals emitted by the nuclear spins are detected by a receiver coil. These RF signals are used to construct the MRI images. These coils can also be referred to as antennas. Further, the transmitter and receiver coils can also be integrated into a single transceiver coil that performs both functions. It is understood that the use of the term transceiver coil also refers to systems where separate transmitter and receiver coils are used.

Using MRI for guiding ultrasonic therapy has the disadvantage that it is not able to detect tumors in all types of tissues. A physician can identify the anatomy of the subject, but may not necessarily know if all of the regions that need to be treated are identified. This is particularly relevant if the tumor has metastasized.

### SUMMARY OF THE INVENTION

Embodiments of the invention provide for a control apparatus for controlling the therapeutic apparatus, a therapeutic apparatus, a method for controlling a therapeutic apparatus, and a computer program product in the independent claims. Embodiments of the invention are given in the dependent claims.

Ultrasound is quickly becoming a desirable approach for specific therapeutic interventions. In particular, the use of High Intensity Focused Ultrasound (HIFU) is currently being used as an approach for thermal therapeutic intervention for uterine fibroids and in the treatment of prostate, liver, brain, and other cancerous lesions. In addition, ultrasound has also been the subject of much research as a means of mediating clot dissolution (sonothrombolysis), localized drug delivery, and gene therapy. The use of ultrasound in all of these applications is desirable because it allows the non-invasive treatment of deep tissues with little or no effect on overlying organs. This leads to reduced systemic side-effects, decreased hospital stay and decreased recovery time. Moreover HIFU can be performed repeatedly and can be used in conjunction with other therapies.

MR spectroscopy is a technique capable of detecting metabolic markers of various tumors and other conditions such as ischemia, trauma, infection, and inflammation. Different chemicals in the same nucleus such as H1, Na23, P31, or F19 exhibit different chemical shifts in resonance frequency that can be exploited to allow the chemical to be identified. Using ¹H, several molecules such as choline, citrate, creatine, water and lipids can be studied. With the advent of efficient water and lipid suppression techniques, ¹H spectroscopy can provide information on other molecules that can be linked to tumor activity. For instance, in the case of prostate adenocarcinoma, a high choline level indicates higher activity of tumor since choline is needed for cell membrane composition. Citrate is necessary for normal prostate metabolism and is decreased in prostate cancer. Creatine, whose peak is located close to choline on the spectra show little difference between normal tissue and prostate cancer. Hence, a commonly used metric to gauge the prostate cancer activity is the ratio: (choline+creatine)/citrate. A small value of this ratio is indicative of normal tissue and a high value is indicative of tumor. Significant improvements in tumor detection is achieved when such MR spectroscopic information is used for prostate cancer treatment. The addition of MR spectroscopic imaging to MR imaging has been demonstrated to lead to a significant increase in the accuracy (52% to 75%) and specificity (26% to 66%) of tumor detection in the prostate.

The ratio of choline peak amplitude to noise amplitude has been used as a measure of malignant lesion in nonmass breast lesions. The study found that MR spectroscopy had a sensitivity and specificity of 100% and 85% respectively in identifying malignant cases in the breast. In another study with 9 patients, it was shown that a significantly higher choline SNR for malignant breast lesions compared to benign lesions.

In the case of brain tissues, the common metabolites include NAA (N-acetyl aspartate), creatine, and choline. As malignancy increases, NAA and creatine decreases and choline levels increase. Low levels of NAA are also indicative of neuronal loss. In a study of musculosketal tumors, proton MR spectroscopic imaging showed significantly higher choline SNR levels for malignant skeletal tumors compared to nonmalignant tissue.

While H-1 is the most common nucleus for MR spectroscopic imaging, other nuclei such as Na23, P31, and F19 can also be used for studying metabolites indicative of a variety of conditions. For example, phosphorus spectra contain information about several metabolites such as ATP and phosphocreatine, phophomonoester, and phosphodiester. Abnormalities in phosphorus metabolites have been shown to be related to tumors, epilepsy and other diseases in the brain. Fluorine MR spectral imaging of TF-MISO have been shown to be indicative of hypoxic tumors.

Shifts in MR spectra also occur due to changes in tissue temperature a phenomenon that is well documented in literature and is the basis of commercial products treating uterine fibroids with MR monitoring.

Embodiments of the invention use MR spectroscopic imaging to guide localized ultrasound therapy, monitor the therapy through temperature changes in the tissues in the case of ablative ultrasound, and optionally evaluate post-operatively the viability of tissues.

Ultrasound based ablation or localized drug delivery can be an effective technique to non-invasively treat specific regions of tissue. However, the placement of the therapeutic zone is not always apparent in most commonly used imaging techniques. Although standard MRI scans can provide detailed anatomic information, there is often little correlation to tumor biology. For example, there is currently no clinically established method to provide targeted therapies to the prostate. This is because prostate cancer is a multi-focal disease and does not readily show up on conventional imaging scans. Current HIFU treatments seek to use ultrasound guidance to destroy the whole prostate. Whole prostate treatment inevitably increases the chance of collateral damage to the neurovascular bundle leading to unwanted impotence. Additionally it leads to increased treatment as well as planning time. This is despite the fact that HIFU can be made to precisely control the location.

This invention solves this problem by locating the distribution of the tumor or other conditions through the use of MR spectroscopy. The combination of ultrasound therapy and MR spectroscopic imaging in a registered integrated device will allow for both immediate treatment of the detected zone but also potentially for a means of measuring treatment outcome. Having an integrated system rather than separate MR spectroscopy and ultrasound therapy systems is highly advantageous for a number of reasons including enabling the registration of the MR spectroscopy data with the ultrasound therapy system, enabling synchronization between imaging and therapy especially when repeated therapy and imaging operations are done, simplification of the user interface for the clinician, and improved workflow in the clinical environment, and avoidance of repeated patient visits.

Magnetic Resonance Imaging (MRI) data is defined herein as being the recorded measurements of radio frequency signals emitted by atomic spins by the antenna of a Magnetic resonance apparatus during a magnetic resonance imaging scan. A Magnetic Resonance Imaging (MRI) image is defined herein as being the reconstructed two or three dimensional visualization of anatomic data contained within the magnetic resonance imaging data. This visualization can be performed using a computer.

Magnetic Resonance (MR) spectroscopic data is defined herein as being the recorded measurements of radio frequency signals emitted by atomic spins by the antenna of a Magnetic resonance apparatus during a magnetic resonance spectroscopic scan. A computer or processor can be used to reconstruct magnetic resonance spectra from the magnetic resonance spectroscopy data. The magnetic resonance spectra are associated with an anatomical region of the subject. The subject may be a mammal. However the resolution is not great enough to construct an image. A Magnetic Resonance (MR) spectroscopy map is defined herein as being a mapping which associates the magnetic resonance spectrums with an anatomical region. In embodiments of the invention the magnetic resonance spectroscopy maps are associated with different regions of a magnetic resonance imaging image.

Magnetic Resonance (MR) is also known as Nuclear Magnetic Resonance (NMR). A Magnetic Resonance apparatus is defined herein as an apparatus that uses magnetic resonance to acquire three dimensionally resolved magnetic resonance imaging data and/or three dimensionally resolved magnetic resonance spectroscopic data.

Magnetic resonance imaging data and magnetic resonance spectroscopy data is typically acquired in slices. Or if the data was acquired as a three dimensional volume the data is typically displayed as a slice. A slice is defined herein as a two dimensional plot showing a magnetic resonance imaging image or a magnetic resonance spectroscopy map for a thin three dimensional slab. It should be noted that the magnetic resonance imaging images are reconstructed using Fourier analysis, so regions of the subject outside the slice contribute to it.

Magnetic resonance thermometry is defined herein as being the non invasive measurement of a region of a subject using magnetic resonance imaging and is based upon the effect of temperature on the physical parameters that affect the MRI or NMR signal. This can for example be performed by measuring the chemical shift of water, measuring the change in the spin lattice relaxation time (T1), or by using diffusion imaging techniques.

A therapeutic ultrasound system is defined herein as being an apparatus capable of emitting ultrasound and concentrating it in a treatment zone such that the ultrasound concentrated in this region is useful for a therapy. At large power levels cavitation can be induced resulting in damage to tissue in the treatment zone. At a power level less than what induces cavitation, the ultrasound can be used to heat tissue. This can be used for tissue ablation and it can be used for treating tumors. Cancerous tissue is less vascularized than healthy tissue and can be effectively destroyed with ultrasonic therapy. The ablation of tissue and destruction of tumors using ultrasound is referred to as High Intensity Focused Ultrasound (HIFU). At powers lower than those used for HIFU or using pulsed power regions of cells can be disrupted using ultrasound. This allows the targeted delivery of genetic material or medicine to cells within the treatment zone.

Ultrasound imaging data is defined herein as being the recorded measurements of ultrasound recorded by a transducer during an ultrasonic examination with an ultrasound imaging system or a therapeutic ultrasound system. An ultrasound image is defined as being the reconstructed two or three dimensional visualization of the ultrasonic imaging data. This visualization can be performed using a computer.

Embodiments of the invention provide for a control apparatus for controlling a therapeutic apparatus. The control apparatus comprises an ultrasound control interface for controlling a therapeutic ultrasound system. The ultrasound control interface is adapted for sending and receiving instructions from a therapeutic ultrasound system. The control apparatus further comprises a magnetic resonance control interface for controlling the magnetic resonance apparatus that is adapted for acquiring magnetic resonance imaging data from a subject and for acquiring magnetic resonance spectroscopy data from the subject. As with the ultrasound control interface, the magnetic resonance control interface is adapted for sending and receiving data from the magnetic resonance apparatus. The control apparatus further comprises an image processing module for generating at least one magnetic resonance image from the magnetic resonance imaging data. The imaging process module is adapted for generating at least one magnetic resonance spectroscopy map from the magnetic resonance spectroscopy data. The image processing module can be a single software module or it can be a collection of software modules adapted for producing image processing. The control apparatus further comprises a planning module adapted for receiving magnetic resonance imaging image and magnetic resonance spectroscopy map and then for outputting planning data. The planning data is the data used by a control module which is adapted for controlling the therapeutic ultrasound system. The control apparatus further comprises a control module adapted for controlling the therapeutic ultrasound system using ultrasound control apparatus using the planning data. The control module is further adapted for controlling the acquisition of magnetic resonance imaging data and magnetic resonance spectroscopy data using the magnetic resonance control interface.

The control apparatus can be a single control system or it can be a collection of controllers or processors. For example, in one embodiment the control apparatus is a single computer system which controls all functions of the therapeutic apparatus. In another embodiment there is a control computer that an operator uses and then separate controllers or computers for individual functions. For example there may be a computer or a collection of computers which performs image processing, there may be a separate computer for controlling the therapeutic ultrasound system, there may be a separate computer or controller for controlling different functions of the magnetic resonance control interface, and finally there may be a separate controller or computer for a planning module. Individual sections of the control apparatus can communicate via a computer network or via a data interface.

In another embodiment, the planning module comprises a graphical user interface. The graphical user interface comprises a display adapted for displaying the magnetic resonance imaging and the magnetic resonance spectroscopy map wherein the magnetic resonance imaging and the magnetic resonance spectroscopy map are superimposed. The display can be any suitable device for displaying graphical data; examples would be a computer monitor screen or a projection system. The graphical user interface further comprises an editing interface adapted for receiving selection data from an operator. The selection data indicates at least one region of the displayed magnetic resonance image and the displayed magnetic spectroscopy map to be treated with ultrasound therapy. The editing interface can be implemented in different ways. A touch sensitive screen could be used for an operator to select a region or select a portion of the screen to be treated. A mouse could be used or other pointing and selection device which are typically used for graphical user interfaces for computers. The planning module is further adapted for using the selection data to generate the planning data. The graphical user interface displays the magnetic resonance spectroscopy map and the magnetic resonance imaging image superimposed and this allows an operator to understand the distribution of spectroscopic information and also the anatomy of a subject. An operator can then input regions which will be treated by the ultrasonic system. This arrangement is advantageous, because it allows both magnetic resonance images and magnetic resonance spectroscopy information to be used to plan therapy. The magnetic resonance imaging image shows very good information about the anatomy of a subject, but it does not provide detailed information like chemistry which can be shown using the magnetic resonance spectroscopy map. In some embodiments the data is input completely by an operator. In other embodiments, the control apparatus produces suggested regions to be treated.

In magnetic resonance imaging and magnetic resonance spectroscopy, the data is acquired in slices. The magnetic resonance imaging images and the magnetic resonance spectroscopy maps correspond to the slices. As such an image or a map refers to a three-dimensional slice of the subject. To perform a full planning, the operator would need to look at all the slices of the patient which are being considered. All of the regions which are desired to be treated in the subject are identified in each of the slices.

In another embodiment the display is divided into blocks representing regions to be treated with ultrasonic therapy. The editing interface is adapted for receiving a selection of blocks from the operator, and the planning module is further adapted for generating planning data using the selected blocks. In this embodiment, the display is divided into regions which can be selected by an operator using the graphical user interface. This embodiment is advantageous, because the resolution of the magnetic resonance spectroscopy is much larger than the magnetic resonance imaging. The magnetic resonance spectroscopy map therefore identifies individual regions which may be necessary to treat with the ultrasonic therapy. The blocks can be of different shapes. They can be square, rectangular, hexagonal, or can be other patterns which tile the display.

In another embodiment the editing interface is further adapted to allow the selection of sub-blocks. A sub-block is defined herein as a portion of an individual block. The planning module is further adapted for generating the planning data using the selected blocks and the selected sub-blocks. This embodiment is advantageous, because during the course of displaying the data it may be apparent that the region to be treated is near a sensitive anatomical structure such as a membrane or the boundary of an organ. By selecting sub-blocks an operator is able to avoid damage to such a sensitive region. The selection of a sub-block can be performed in several different ways. There can be a button on the user interface which changes the resolution of the blocks and allows the operator to select sub-blocks. Another possibility is that the user draws or otherwise selects a geometrical region using a mouse, pointer or other device for interacting with the graphical user interface.

In another embodiment, the ultrasound control system is adapted for controlling a therapeutic ultrasound system capable of performing both ultrasound therapy and acquiring ultrasound imaging data. The image processing module is further adapted for generating at least one ultrasound image from the ultrasound imaging data. The planning module is further adapted for receiving ultrasound imaging data. This embodiment is advantageous, because ultrasound imaging data can be acquired and ultrasound images can be computed which can be used to assist in targeting tissue regions during the use of the therapeutic ultrasound system. This can be implemented in different ways, there can be additional transducers within the therapeutic ultrasound system for performing images, the transceiver for the therapeutic ultrasound system can be operated in an inter-leaved way where imaging is performed alternatively with performing the therapy. Some ultrasonic transducers are adapted such that a portion of the transceiver system can be used for therapy and a portion can be used for imaging.

In another embodiment, the planning module comprises a graphical user interface wherein the graphical user interface comprises a display for displaying the ultrasound image, the magnetic resonance imaging map and the magnetic resonance spectroscopy map. The ultrasound image, the magnetic resonance imaging image and the magnetic resonance spectroscopy map are superimposed. This is advantageous because the ultrasound image can show additional anatomical data. In the case of performing a cancer treatment, a subject can be injected with contrast agents which becomes visible through ultrasound, so in all three modalities on the same screen allows better decisions to be made during the planning of the therapy. The editing interface is adapted for receiving selection data from an operator. The selection data indicates at least one region of the displayed ultrasound image, and the displayed image, displayed magnetic resonance imaging and the displayed magnetic resonance spectroscopy map can be treated with the ultrasound therapy. The selection of the regions and of selecting in multiple slices has been described previously. The planning module is adapted for using the selection data to generate the planning data.

In another embodiment, the display is further adapted for displaying a signal from a ultrasound contrast agent. This embodiment is beneficial, because ultrasound contrast agents can be designed such that they concentrate in regions with a specific pathology. For example ultrasound contrast agent can have targeting entities that bond to cancer cells. Ultrasound contrast agents can also accumulate in tumorous leaky vasculature.

In another embodiment the planning module uses selection data that indicates at least one region of the magnetic resonance imaging image and the magnetic resonance spectroscopy map to be treated with ultrasonic therapy to generate the planning data. The planning module comprises a pattern recognition module for generating the selection data. The pattern recognition module can be implemented using standard image segmentation techniques. The pattern recognition module can also be implemented as a trainable pattern recognition module.

A trainable pattern recognition module is defined herein as a pattern recognition module that can be trained using a set of training images comprising at least the magnetic resonance imaging image and the magnetic resonance spectroscopy data. The training images have had at least one region that has been identified for therapy prior to being used for training. A trainable pattern recognition module can be implemented by using a variety of different methods. Examples of different methods or algorithms that could be used are: Principal Component Analysis, Neural Network, CN2 algorithm, C4.5 algorithm, Iterative Dichotomiser 3 (ID3), nearest neighbor search algorithm, naive Bayes classifier algorithm, Holographic Associative Memory, or perception learning algorithm.

This embodiment is advantageous, because segmentation and pattern recognition software can be used to automatically identify regions of a subject to be treated. The system can proceed automatically, or the pattern recognition module can provide a suggested therapy plan for the subject. An operator can simply approve the proposed therapy, or an operator can edit and modify the plan.

In another embodiment, the magnetic resonance control interface is adapted for controlling a magnetic resonance apparatus that is further adapted for performing magnetic resonance imaging thermometry. The ultrasonic control interface is adapted for controlling an ultrasound apparatus adapted for applying ultrasound therapy to a treatment zone. The control module is further adapted for continuously monitoring the temperature of the subject in the region surrounding the treatment zone. The control module is adapted for modifying the planning data in real time based upon the temperature in the region surrounding the treatment zone. This embodiment is advantageous, because in many ultrasonic therapies the goal is to heat the tissue in order to ablate tissue or to kill diseased or cancerous tissue. By monitoring the temperature in the tissue surrounding the treatment zone, better control of the treatment can be maintained.

In another embodiment, the magnetic resonance apparatus is adapted for performing post evaluation treatment evaluation of the subject with magnetic resonance imaging. In one embodiment, the follow-up after HIFU treatment would be to perform MR contrast imaging. Gadolinium enhanced imaging can show the perfused versus non-perfused regions, and the non-perfused region is indicative of the tissues that were destroyed by HIFU. This is advantageous, because the magnetic resonance imaging can be used to determine how effective a treatment was.

In another aspect the invention provides for a therapeutic apparatus. The therapeutic apparatus comprises a control apparatus according to an embodiment of the invention, a therapeutic ultrasound system, and a magnetic resonance apparatus. A therapeutic apparatus according to an embodiment of the invention is advantageous, because the detailed magnetic resonance spectroscopy and magnetic resonance imaging data can be combined to develop an effective treatment plan for the therapeutic ultrasound system. The magnetic resonance apparatus is capable of performing both magnetic resonance imaging and magnetic resonance spectroscopy. Magnetic resonance apparatuses are capable of performing both of these are modified from standard magnetic resonance imaging systems by having different software and also an updated radio frequency system. The coil used by the radio frequency system in some embodiments is a single coil that is capable of doing both magnetic resonance imaging and magnetic resonance spectroscopy. In other embodiments a separate coil is used for magnetic resonance imaging and for magnetic resonance spectroscopy.

In another aspect the invention provides for a method for controlling a therapeutic apparatus. The method comprises acquiring magnetic resonance imaging data with a magnetic resonance apparatus. The method further comprises processing the magnetic resonance imaging data with an imaging process module to generate at least one magnetic resonance image. At this step the magnetic resonance imaging data is turned into an image which can be used for diagnostic purposes. The method further comprises acquiring magnetic resonance spectroscopy data with a magnetic resonance imaging apparatus. The method further comprises processing the magnetic resonance spectroscopy data with the image processing module to generate at least one magnetic resonance spectroscopy map. The method further comprises outputting the magnetic resonance imaging image and the magnetic resonance spectroscopy map to a planning module. In the planning module, data which is needed for controlling a therapeutic ultrasound system is generated. The method further comprises receiving planning data from the planning module. The method further comprises controlling the treatment of a subject with a therapeutic ultrasound system using the planning data. At this step the planning data is used for the treatment of a subject. The advantages of this method have been previously described.

In another embodiment, the method further comprises displaying the magnetic resonance imaging and the magnetic resonance spectroscopy data on a display such as a magnetic resonance imaging image and the magnetic resonance spectroscopy map are superimposed. The advantages of this have been previously discussed. The method further comprises the steps of receiving selection data from an operator using an editing interface. The selection data indicates at least one region of the displayed magnetic resonance imaging and the displayed magnetic resonance spectroscopy map to be treated with the ultrasound therapy. The method further comprises generating the planning data using the selection data.

The method further comprises the steps of registering the location of the subject with the magnetic resonance imaging before acquiring the magnetic resonance spectroscopy data. The method further comprises the step of registering the location of the subject again before commencing the step of controlling the treatment of a subject with the therapeutic ultrasound system. This embodiment is advantageous, because in order to generate the planning data the magnetic resonance imaging data is acquired, the magnetic resonance spectroscopy data is acquired and then detailed plans are made and planning data is generated. It is advantageous to check the location of the subject again to make sure that the subject has not moved, because if the subject has moved it is possible that vulnerable regions such as membranes or the boundaries of organs could be damaged or destroyed. The registration of the location of the subject can be done in several ways. Imaging processing module or controller can be used to segment the image and automatically register the images. It is also possible to put fiducial markers on the surface of a subject. These markers can either contain a substance easily imaged by the magnetic resonance imaging or it can be an antenna which is resonant. Fiducial markers allow easy identification of the location of a subject.

In another embodiment the method further comprises the step of performing post-evaluation treatment evaluation of a subject with magnetic resonance imaging. In one embodiment, the follow-up after HIFU treatment would be to perform MR contrast imaging. Gadolinium enhanced imaging can show the perfused versus non-perfused regions, and the non-perfused region is indicative of the tissues that were destroyed by HIFU. This is advantageous, because the magnetic resonance imaging can be used to determine how effective a treatment was.

In another embodiment, the method further comprises the steps of performing magnetic resonance imaging thermometry and adjusting the planning data with the planning module using the thermometry measurements. The ultrasonic control interface is adapted for controlling an ultrasound apparatus adapted for applying ultrasound therapy to a treatment zone. The control module is further adapted for continuously monitoring the temperature of the subject in the region surrounding the treatment zone. The control module is adapted for modifying the planning data in real time based upon the temperature in the region surrounding the treatment zone. This embodiment is advantageous, because in many ultrasonic therapies the goal is to heat the tissue in order to ablate tissue or to kill diseased or cancerous tissue. By monitoring the temperature in the tissue surrounding the treatment zone, better control of the treatment can be maintained.

In another aspect the invention provides for a computer program product comprising machine executable code for performing the method according to an embodiment of the invention on a control apparatus for a therapeutic apparatus. The advantages of this have been previously discussed.

### BRIEF DESCRIPTION OF THE DRAWINGS

In the following preferred embodiments of the invention will be described, by way of example only, and with reference to the drawings in which:

FIG. 1 shows a functional diagram of a control apparatus according to an embodiment of the invention,

FIG. 2 shows a functional diagram of a therapeutic apparatus according to an embodiment of the invention,

FIG. 3 shows an example of a graphical user interface according to an embodiment of the invention,

FIG. 4 shows an example of a method according to an embodiment of the invention,

FIG. 5 shows an MRI image with an illustration of a MR spectroscopy map,

FIG. 6 illustrates an embodiment of a method of selecting block to be treated with therapeutic ultrasound,

FIG. 7 illustrates the path that a therapeutic apparatus treats in a single block.

### DETAILED DESCRIPTION

Like numbered elements in these figures are either identical elements or perform the same function. Elements which have been discussed previously will not necessarily be discussed in later figures if the function is identical.

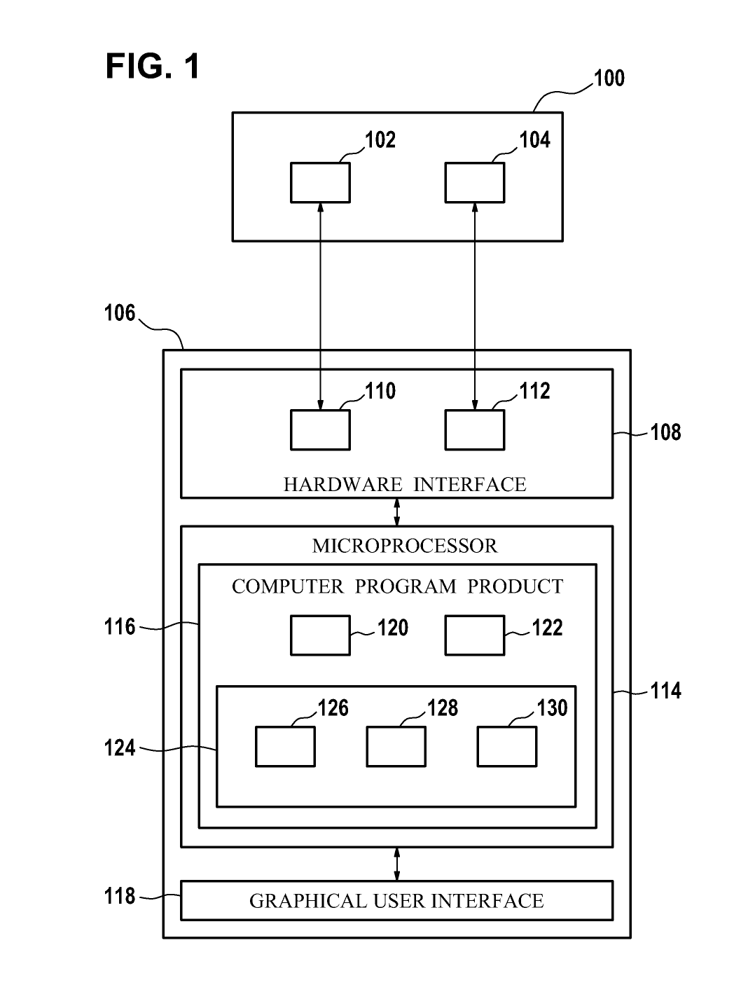

FIG. 1 shows a control apparatus 106 according to an embodiment of the invention. FIG. 1 shows a therapeutic apparatus 100 and a control apparatus 106. The therapeutic apparatus 100 comprises a therapeutic ultrasound system 102 and a magnetic resonance apparatus 104. The control apparatus 106 has a hardware interface 108 that is for interfacing to external hardware. The hardware interface 108 is able to send and receive data. The hardware interface 108 has a sub-component which is an ultrasound control interface 110 which is connected to the therapeutic ultrasound system 102.

The hardware interface 108 also has a magnetic resonance control interface 112 which is connected to the magnetic resonance apparatus 104. The hardware interface is connected to a microprocessor 114. The microprocessor 114 is representative of any processing unit able to perform instructions in order to control the therapeutic apparatus. Examples would be a microprocessor, a controller, or an embedded system. There is a computer program product 116 comprising machine executable code which is able to be executed on the microprocessor 114.

The computer program product comprises different executable instruction components or modules for performing different tasks. The computer program product comprises a control module 122. The control module is able to control the therapeutic ultrasound system 102 through the ultrasound control interface 110 and is also able to control the magnetic resonance apparatus 104 through the magnetic resonance control interface 112. Essentially the control module 122 controls the acquisition of data and it also controls the operation of the therapeutic apparatus during therapy. The computer program product also comprises an image processing module 124. The image processing module is able to take raw magnetic resonance imaging data and magnetic resonance spectroscopy data and turn these into magnetic resonance imaging images and magnetic resonance spectroscopy maps.

In some embodiments the image processing module also comprises a component which is able to take ultrasound data and construct an ultrasound image. In practice these can be separate software components or the components of the image processing module 124 can be a single module. In this figure the image processing module is shown as comprising an image processing module for generating magnetic resonance imaging images 126, an image processing module for generating magnetic resonance spectroscopy maps 128, and an image processing module for generating ultrasound images 130.

The control apparatus 106 also comprises a graphical user interface 118. The graphical user interface 118 is able to interact with the computer program product 116 which runs on a microprocessor 114. The graphical user interface 118 is adapted for displaying the medical imaging images such as the magnetic resonance spectroscopy map and the magnetic resonance imaging image in a format which an operator is able to understand. The graphical user interface is also adapted for receiving selections from an operator. In some embodiments where the computer program product also comprises a pattern recognition module for automatic planning, the graphical user interface can also comprise a means for displaying a suggested therapeutic operation and possibly a means for an operator to edit it or to approve or disapprove of a treatment plan.

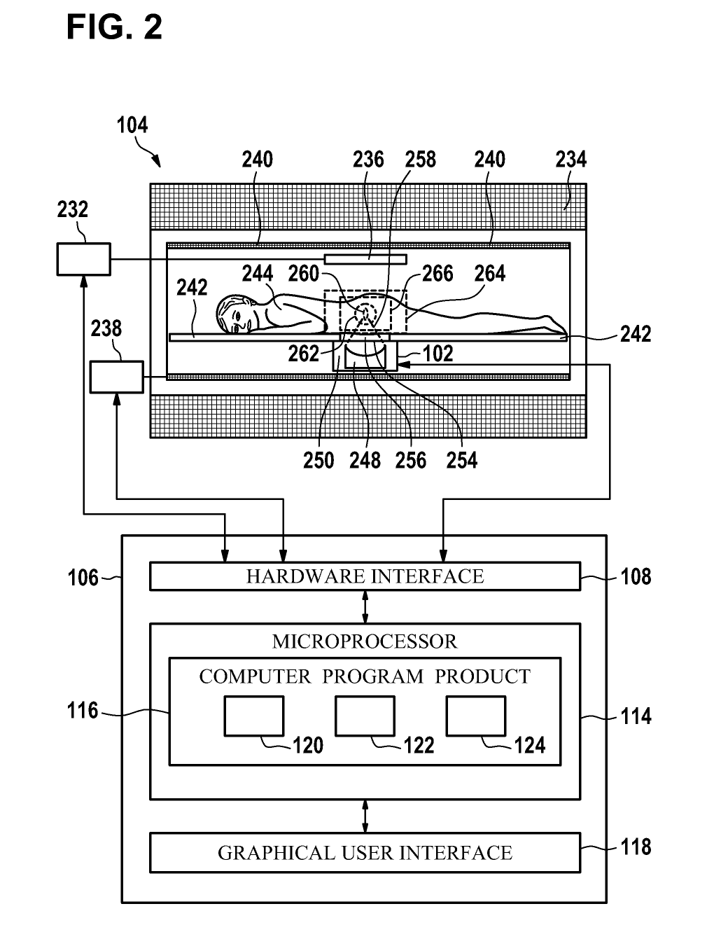

FIG. 2 shows a cross-sectional schematic diagram of a therapeutic apparatus according to an embodiment of the invention. There is a therapeutic ultrasound system 102 that is located within a magnetic resonance apparatus 104. The therapeutic ultrasound system 102 and the magnetic resonance apparatus 104 are controlled by a control apparatus 106. The therapeutic ultrasound system 102 is connected to the hardware interface 108 of the control apparatus 106. The hardware interface is also connected to a magnetic field gradient power supply 238. The magnetic field gradient power supply 238 is adapted for powering the magnetic field gradient coil assembly 240. The magnetic resonance apparatus comprises a magnet 234 which generates a magnetic field which is capable of polarizing the atomic spins of atoms within an imaging zone 264 for magnetic resonance imaging. The embodiment of the magnet 234 shown here is a cross-sectional view of a cylindrical magnet. The magnet can be constructed using permanent magnets, electromagnets, a superconducting magnet, or a combination of all three or just two. Other configurations of magnets besides cylindrical are also possible. The magnetic field gradient power supply energizes the magnetic field gradient coil assembly and creates a magnetic field which is able to add spatial encoding to the location of atomic spins within the imaging zone 264. The hardware interface 108 is also connected to a radio frequency transceiver 232. The radio frequency transceiver is connected to a radio frequency coil 236. The radio frequency coil produces radio frequency transmissions which are able to manipulate the orientation of atomic spins within the imaging zone 264. As atomic spins relax they emit radio frequency transmissions which are received by the radio frequency coil 236. In some embodiments there are separate transmit and receive coils. It is understood that both possibilities are possible in this example only the dual purpose radio frequency coil is shown 236. For acquiring magnetic resonance spectroscopy data and for acquiring magnetic resonance imaging data it is possible to use separate radio frequency coils 236 also. However, as is shown in this embodiment it is also possible to use a single coil for both purposes. Within the magnet 234 there is a patient support 242 which is capable of supporting a subject 244. The need for the patient support 242 is the therapeutic ultrasound system 102. The therapeutic ultrasound system 102 comprises an ultrasonic transducer 248. The ultrasonic transducer is located typically inside of a chamber filled with a ultrasonic ducting medium 250. Typically the chamber 250 is filled with the gas/water or some other material capable of transmitting ultrasound. In many embodiments the ultrasound transducer is capable of being moved slightly to change the focus of the ultrasound within the subject 244. There is an ultrasonic membrane 254 which is adapted for transmitting ultrasound. This seals the chamber 250 which contains the ultrasonic transducer 248. Between the membrane 254 and the subject 244 there is typically a cavity which is adapted for receiving an ultrasonic coupling medium 256. The ultrasonic coupling medium can be water, it can be an ultrasonic gel, or it can be a gel pad. The purpose of using an ultrasonic coupling medium is that if there are any air bubbles or any air spaces in the path of the ultrasound to a treatment zone 260, the subject can receive burns. The ultrasound leaves the ultrasound transducer 248 and follows a path 258 through the subject 244 to a treatment zone 260. In the treatment zone different things can occur, for very large powers, ablation of tissue or heating of tissue to the point of tissue necrosis can occur. Beneath the radio frequency coil 236 there is a region 264 where magnetic resonance imaging data can be acquired. The magnetic resonance spectroscopy data typically takes more time to acquire than magnetic resonance imaging data. So typically a physician would acquire the magnetic resonance imaging data 264 and then locate the relevant anatomy of the subject 244 and decide on a region 266 for which to perform the magnetic resonance spectroscopy. During the actual therapy it may be advantageous to use magnetic resonance thermometry to monitor the temperature of tissue surrounding the treatment zone 260. In the figure there is a region 262 surrounding the treatment zone 260. This is representative of a region to be used to measure the temperature during the actual therapeutic operation.

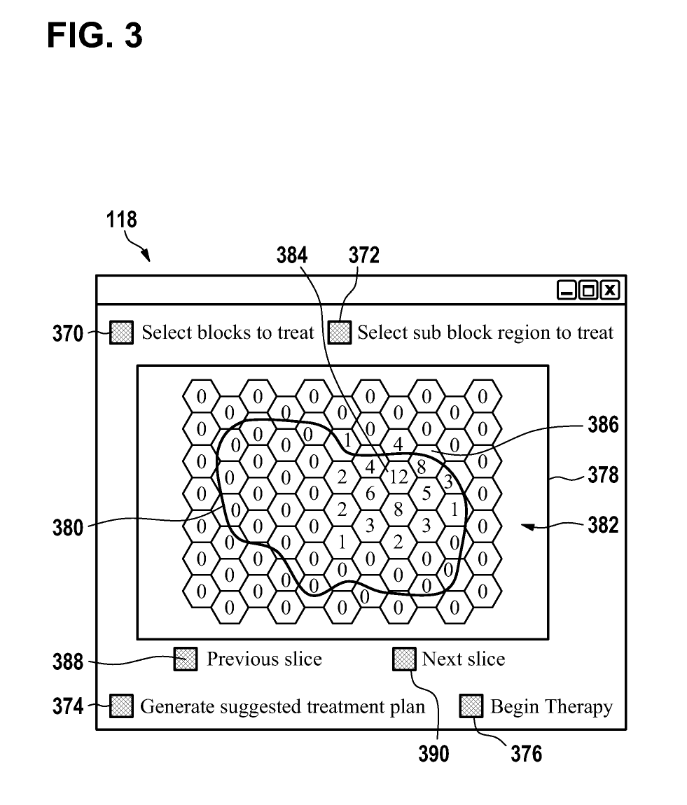

FIG. 3 shows an example of an embodiment of a graphical user interface 118 according to an embodiment of the invention. There is a display 378 which is capable of displaying both magnetic resonance imaging data and magnetic resonance spectroscopy data. The curve 380 represents the boundary of an organ which is shown in a magnetic resonance imaging image. There are hexagons 382 also shown in the display 378. An operator is able to click any one of these hexagons and then this region will be in the region that is treated with ultrasound therapy. There are numbers within each of these hexagons. These represent an arbitrary measure of some magnetic resonance spectroscopy map. When an operator looks at the display 378 he or she can see the anatomy from the magnetic resonance imaging image 380 and then look at the display and see likely which area needs therapy. In some embodiments the magnetic resonance spectroscopy map can be shown in different ways. In this embodiment it is shown by numbers, it can also be color or grayscale encoded. Hexagon 384 shows a value of 12. In this case it is very likely that the physician would probably want to treat this region. So in this case the operator could simply click on this hexagon and this would be added to the treatment plan. Hexagon 386 is a different matter. The boundary of the organ 380 runs right through this hexagon. It is likely that an operator or physician would want to not treat the entire region. In this case the physician or operator clicks button 372. Then the physician is able to draw a sub-region that he or she would like to treat and is thus able to avoid destroying the boundary of the organ. There is a button 370 which allows the operator to go into a mode where entire blocks are able to be treated. Button 372 allows a sub-block to be selected. Button 374 causes a pattern recognition module to generate a suggested treatment plan. The operator can then modify the treatment plan using the graphical user interface 118. When the user is satisfied with the therapy he or she can click button 378 and then planning data is generated by the planning module. The magnetic resonance imaging data and the magnetic resonance spectroscopy data are acquired in slices. To do a complete treatment plan, all the slices of interest need to be examined. Button 388 and button 390 allow an operator to select which slice to examine. The blocks 382 shown in this example are hexagonal. The blocks can also be square or rectangular. They do not need to have a specific shape but these examples are chosen because they are uniform and the region treated with the therapeutic ultrasound approximates a sphere which is well approximated by a hexagon in one cross-section.

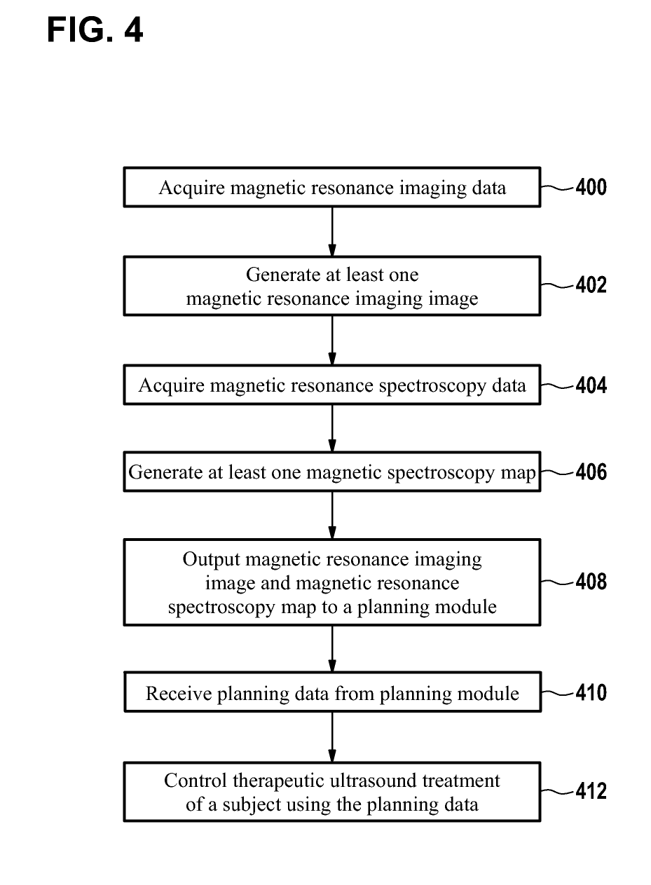

FIG. 4 shows an example of a method according to an embodiment of the invention. In step 400 magnetic resonance imaging data is acquired. In step 402 the magnetic resonance imaging data is used to generate at least one magnetic resonance imaging image. In step 404 magnetic resonance spectroscopy data is acquired. In step 406 at least one magnetic resonance spectroscopy map is generated using the magnetic resonance spectroscopy data. In step 408 the magnetic resonance imaging image and the magnetic resonance spectroscopy map are output to a planning module. In step 410 the planning data is received from the planning module. In step 412 the planning data is used by a control module to control the therapeutic ultrasound treatment of a subject.

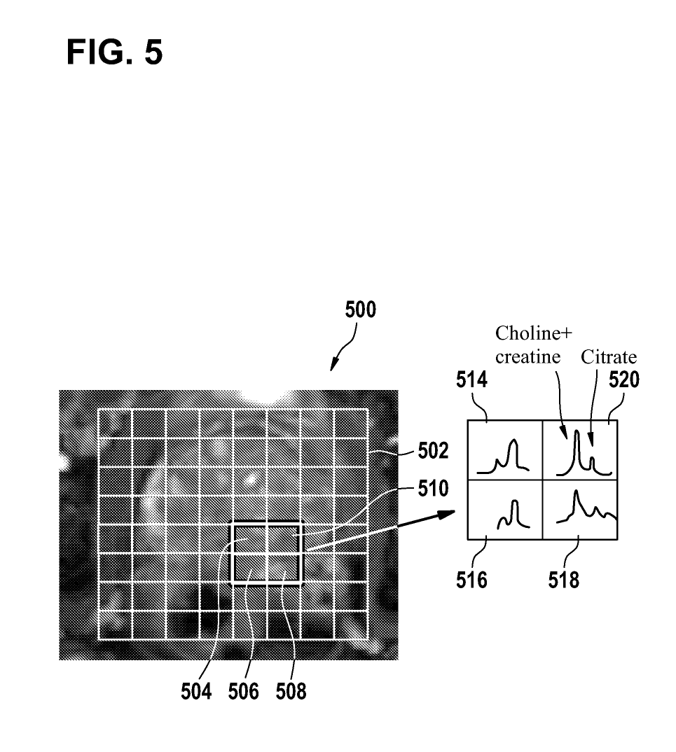

FIG. 5 shows an illustration of a combined magnetic resonance imaging image 500 and a grid containing a magnetic resonance spectroscopy map 502. Within each of the squares 502 there is an associated magnetic resonance spectrum. A detailed magnetic resonance spectrum is shown for four of these squares labeled 504, 506, 508 and 510. Spectroscopic data 514 corresponds to square 504. Spectroscopic data 516 corresponds to square 506. Spectroscopic data 520 corresponds to region 510. Spectroscopic data 518 corresponds to region 508. Spectroscopic data 518 and 520 show high choline plus creatine levels compared to the citrate level. This is indicative of a tumor.

FIG. 5 illustrates the use of an embodiment where an ultrasound transducer consisting of one or more elements for therapy is seated in a tissue-coupling medium. The transducer is registered in the coordinate system of the MR imaging system. The MR imaging system captures a volumetric image of the pathological tissue. MR spectroscopy (MRS) information based on proton resonance is obtained, preferably in 3D. The ratio of (choline+creatine)/citrate is obtained and this information is overlaid as a grid pattern on top of the MR images (cf. FIG. 5). High values of this ratio are color coded to indicate higher probability of tumor presence and aggressiveness. Both the MR images and MRS data are sent to the planning console. The MRS data are shown as an overlay on the MR image.

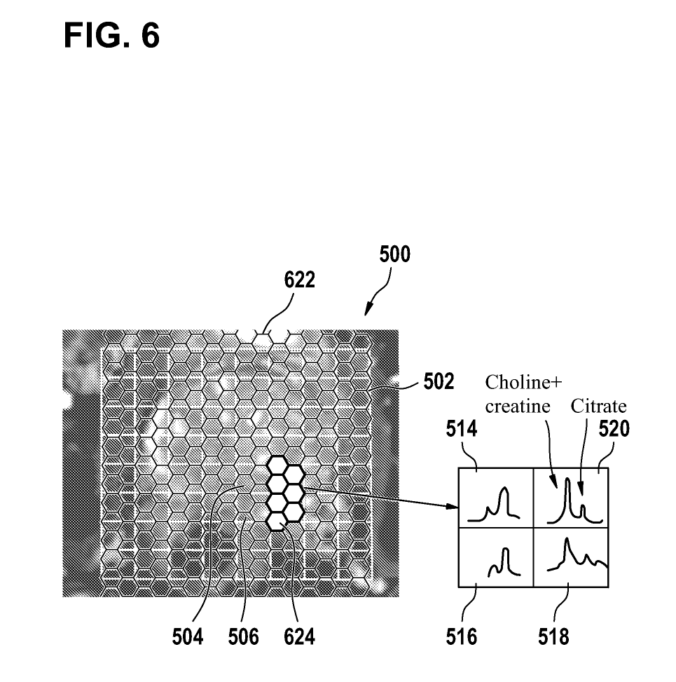

FIG. 6 is an illustration of how an operator would use the information presented in FIG. 5 to select regions to treat using ultrasonic therapy. FIG. 6 shows the same information as was shown in FIG. 5. On top of FIG. 5 has been superimposed a latticework of hexagonal blocks 622 which indicate distinct regions which can be treated with ultrasonic therapy. Item 624 is a group of seven hexagonal blocks which have been selected for therapy. These blocks cover the regions where region 510 and 508 were previously. These were the two regions that were indicated by the MR spectrum in 520 and 518 as likely containing a tumor. These regions would be used to generate the planning data event.

FIG. 6 illustrates how an operator confirms the region to be treated and activates a therapy planning hexagonal grid onto the images (FIG. 6). The system spatially maps the spectroscopy grids that are indicative of therapy to the treatment cells formed by the therapy grid. The operator can also adjust the therapy levels (ultrasound power and/or duration and/or duty cycle) to the therapy regions suggested depending on combined MR image and MRS data presented. The operator then activates the therapy. Through co-registration of the ultrasound therapy system with the MR imaging coordinate system, the therapeutic ultrasound is delivered to the specific diseased tissue, one therapy grid at a time. The treatment within a particular treatment cell can be done through point-by-point scanning or a volumetric scan pattern. The therapeutic ultrasound destroys the selected tissue through ablation. MR based temperature and thermal dose distribution data are obtained at intermittent intervals to provide direct feedback to further adjust the therapy.

In another embodiment, the ultrasound therapy transducer (e.g. HIFU transducer) can be an array with a large number of elements placed in the rectum of the patient directed towards the prostate. The array can be a 1D, 1.5D, or a 2D array with capability for rotation to cover various regions in the prostate. The planning console calculates the delays for each element in order to focus the therapy beam onto the selected region based on MRS data.

In another embodiment, the therapy transducer is adapted to be placed with a few elements in the urethra with direct access to the prostate. The ultrasound transducer can be rotated or translated if needed to cover specific regions identified by MRS data.

In an embodiments, Spectroscopic information is used to obtain changes in tissue temperatures in order to determine regions that have been coagulated or ablated. Such information is then used to stop or modify the therapy, e.g., through adjustments to the power to the therapy transducer.

In another embodiment, the therapy transducer is placed outside the patient's body (e.g. for breast or liver applications). In breast applications the aim would be to identify and ablate malignant tissues and leave out benign tissues.

In another embodiment, ultrasound is used to provide localized delivery of drugs or genetic material. In this embodiment, an injection or infusion of acoustically active agents (microbubbles or perfluorocarbon nanoparticles) is administered to the patient before ultrasound is activated. These agents can optionally have targeting mimetics to bind to specific sites in the body, which can enhance the information provided by MRS data. In this embodiment, the temperature imaging may not be used.

In another embodiment, one or more nuclei other than H-1 or in addition to H-1 (e.g. P-31, Na-23, or F-19) to obtain metabolic information for guiding therapy and/or obtaining changes in tissue temperature.

In another embodiment, multi-parametric imaging where MR spectroscopy is combined with diffusion imaging and dynamic contrast imaging to obtain tissue signatures indicative of the conditions being assessed.

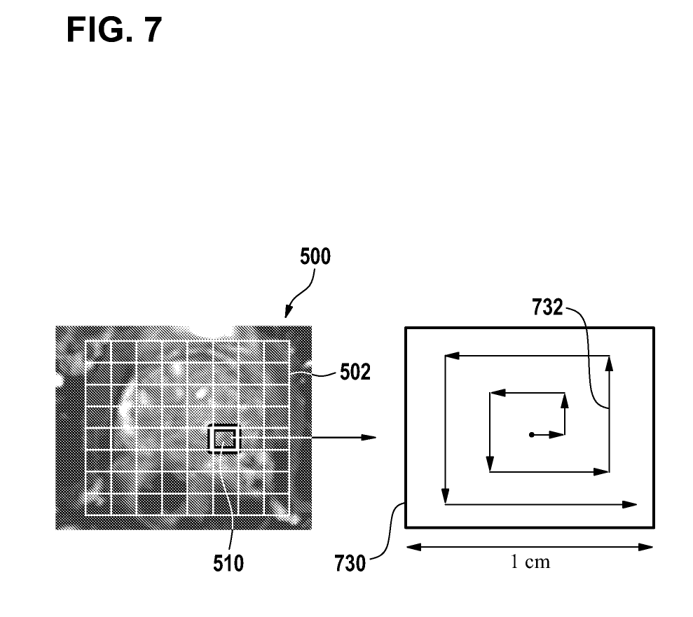

FIG. 7 shows the alternative way of generating the planning data. The magnetic resonance imaging data 500 and magnetic resonance spectroscopy map 502 from FIG. 5 is shown in FIG. 7. The element 730 represents the region 510 in the magnetic resonance imaging image 500. In order to ensure a more uniform heating within the treatment square and prevent damage outside, path 732 shows that the ultrasound beam traverses a path from the center and moves outwards.

MR spectroscopy provides tissue specific information within a square grid. The squares are typically much larger than the MR image resolution and can be about 1 cm in size. The ultrasound therapy beam size in the focal plane (1 mm) is much smaller than the spectroscopy grid size. The therapy beam must therefore be steered in order to cover the treatment spectroscopy grid. The therapy plan would depend on the particular characteristics of the ultrasound used, namely the frequency of operation, aperture size, and the location of the focal spot. A lower frequency of operation would imply a larger beam size and therefore smaller number of points to traverse within the square. A simple point-by-point scan approach would take significant time and would lead to unintended damage beyond the spectroscopy grid due to heat diffusion. In order to ensure a more uniform heating within the treatment square and to prevent damage outside, the ultrasound beam should traverse a path from the center and move outwards. One such path is a series of squares of increasing size starting from the inside and moving towards outside as illustrated in FIG. 7.

Another possible treatment scheme is shown in FIG. 6. Here the MR spectroscopy square is split into several treatment cells, depicted as hexagonal cells. The treatment proceeds from one cell to the next. There are multiple sonification points within each treatment cell to cover the hexagonal shape. For instance, a series of concentric circles starting from the inside to the outside can be used for the ultrasound therapy beam path within a cell. Once a cell is treated, the next one is treated and so on to cover the square.

Modulation of the ultrasound therapy based on the MR spectroscopy values: There are several reasons to adjust the settings of the ultrasound therapy from one treatment square to the next, or within each square itself. The data from MR spectroscopy indicate a score for the presence of tumor. There would often be regions where the computed scores are neither close to that of healthy tissue nor close to that of malignant tissue. In addition there could be critical tissues that are present close to such treatment square, e.g, nerves or major blood vessels, which need to be preserved. In such cases the operator would prefer to use higher frequency of sonification in order to reduce the beam size and treat only parts of the square that are away from the critical organs. This would ensure that the critical organs are preserved. Such a procedure would increase the accuracy and safety of the procedure at the cost of increased treatment duration. Such a modulation scheme is not obvious from the prior art.

Applications of the Invention

Conditions that be treated with the present invention include tumors, ischmeia, infarct, localized injuries and trauma, inflammation, and infection.

In the case of tumor ablation, applications include the breast, prostate, liver, brain, skeletal and bone tumors. The invention can be used along with other treatments, especially for patients where more invasive treatments such as radiation have failed. Treatments can be repeatedly performed if so desired.

In addition to tumor ablation, the invention can be used in delivery of drugs or genes to the tumors locally, or using ultrasound to sensitize tissues for other treatments.

### LIST OF REFERENCE NUMERALS

| | |
|---|---|
| 100 | Therapeutic apparatus |
| 102 | Therapeutic ultrasound system |
| 104 | Magnetic resonance apparatus |
| 106 | Control apparatus |
| 108 | Hardware interface |
| 110 | Ultrasound control interface |
| 112 | Magnetic resonance control interface |
| 114 | Microprocessor |
| 116 | Computer program product |
| 118 | Graphical user interface |
| 120 | Planning module |
| 122 | Control module |
| 124 | Image processing module |
| 126 | Image processing module for |
| | generating magnetic resonance |
| | imaging images |
| 128 | Image processing module for |
| | generating magnetic resonance |
| | spectroscopy maps |
| 130 | Imaging processing module for |
| | generating ultrasound images |
| 232 | Radio frequency transceiver |
| 234 | magnet |
| 236 | Radio frequency coil |
| 238 | Magnetic field gradient coil power |
| | supply |
| 240 | Magnetic field gradient coil assembly |
| 242 | subject support |
| 244 | subject |
| 248 | Ultrasonic transducer |
| 250 | Chamber filled with ultrasound |
| | transmitting fluid |
| 254 | Ultrasound transmitting membrane |
| 256 | Cavity adapted for receiving an |
| | ultrasonic coupling medium |
| 258 | path of ultrasound |
| 260 | Treatment zone |
| 262 | Region surrounding treatment zone |
| 264 | Zone where magnetic resonance |
| | imaging data is acquired |
| 266 | Zone where magnetic resonance |
| | spectroscopy data is acquired |
| 370 | Select blocks to treat button |
| 372 | Select sub block region to treat button |
| 374 | Generate suggested treatment plan |
| | button |
| 376 | Begin therapy button |
| 378 | Display |
| 380 | Boundary of organ |
| 382 | Blocks |
| 384 | block with a value of 12 |
| 386 | Block with a value of 8 and the |
| | boundary of membrane through it |
| 388 | Previous slice button |
| 390 | Next slice button |
| 500 | Magnetic resonance imaging image |
| | of prostate |
| 502 | Squares showing boundaries of |
| | magnetic resonance spectroscopy map |
| 504 | Region where MR spectrum is shown |
| | in plot 514 |
| 506 | Region where MR spectrum is shown |
| | in plot 516 |
| 508 | Region where MR spectrum is shown |
| | in plot 518 |
| 510 | Region where MR spectrum is shown |
| | in plot 520 |
| 514 | MR spectrum for region 504 |
| 516 | MR spectrum for region 506 |
| 518 | MR spectrum for region 508 |
| 520 | MR spectrum for region 510 |
| 622 | Hexagonal blocks indicating regions |
| | to treat with ultrasonic therapy |
| 624 | Hexagonal blocks where therapy has |
| | been selected |
| 730 | Region representing block 510 |
| 732 | Path followed by ultrasonic treatment |

1. A control apparatus (106) for controlling a therapeutic apparatus (100), wherein the control apparatus comprises:
an ultrasound control interface (110) for controlling a therapeutic ultrasound system (102),
a magnetic resonance control interface (112) for controlling a magnetic resonance apparatus (104) adapted for acquiring magnetic resonance imaging data from a subject and for acquiring magnetic resonance spectroscopy data from a subject (244),
an image processing module (124, 126, 128) for generating at least one magnetic resonance imaging image (500) from the magnetic resonance imaging data and for generating at least one magnetic resonance spectroscopy map (502, 514, 516, 518, 520) from the magnetic resonance spectroscopy data,
a planning module (120) adapted for receiving the magnetic resonance imaging image and the magnetic resonance spectroscopy map and for outputting planning data (732),
a control module (122) adapted for controlling the therapeutic ultrasound system using the ultrasound control apparatus using the planning data, wherein the control module is further adapted for controlling the acquisition of the acquiring magnetic resonance imaging data and magnetic resonance spectroscopy data using the magnetic resonance control interface.

2. The control apparatus of claim 1, wherein the planning module comprises a graphical user interface (118), wherein the graphical user interface comprises:
a display (378) adapted for displaying the magnetic resonance imaging image (380) and the magnetic resonance spectroscopy map (382), wherein the magnetic resonance imaging image and the magnetic resonance spectroscopy map are superimposed,
an editing interface (370, 372, 274, 376, 382, 388, 390, 622, 624) adapted for receiving selection data from an operator, wherein the selection data indicates at least one region (624) of the displayed magnetic resonance image and the displayed magnetic spectroscopy map to be treated with ultrasonic therapy, and
wherein the planning module is adapted for using the selection data to generate the planning data.

3. The control apparatus of claim 2, wherein the display is divided into blocks (382, 502, 622, 624) representing regions to be treated with ultrasonic therapy, and wherein the editing interface is adapted for receiving the selection of blocks from the operator, and wherein the planning module is further adapted for generating the planning data using the selected blocks.

4. The control apparatus of claim 3, wherein the editing interface is further adapted to allow the selection of sub blocks (372), wherein a sub block is a portion of an individual block, and wherein the planning module is further adapted for generating the planning data using the selected blocks and the selected sub blocks.

5. The control apparatus of claim 1, wherein the ultrasound control system is adapted for controlling a therapeutic ultrasound system capable of performing both ultrasound therapy and acquiring ultrasound imaging data, wherein the image processing module (124, 130) is further adapted for generating at least one ultrasound image from the ultrasound imaging data, wherein the planning module is further adapted for receiving ultrasound imaging data.

6. The control apparatus of claim 5, wherein the planning module comprises a graphical user interface (118), wherein the graphical user interface comprises:
a display (378) for displaying the ultrasound image, the magnetic resonance imaging image, and the magnetic resonance spectroscopy map, wherein the ultrasound image, the magnetic resonance imaging image, and the magnetic resonance spectroscopy map are superimposed,
an editing interface (370, 372, 274, 376, 382, 388, 390, 622, 624) adapted for receiving selection data from an operator, wherein the selection data indicates at least one region of the displayed ultrasound image, the displayed magnetic resonance image, and the displayed magnetic spectroscopy map to be treated with ultrasonic therapy, and
wherein the planning module is adapted for using the selection data to generate the planning data.

7. The control apparatus of claim 5, wherein the display is further adapted for displaying a signal from an ultrasound contrast agent.

8. The control apparatus of claim 1, wherein the planning module uses selection data that indicates at least one region (624) of the magnetic resonance image and the magnetic spectroscopy map to be treated with ultrasonic therapy to generate the planning data, wherein the planning module comprises a pattern recognition module for generating the selection data.

9. The control apparatus of claim 1, wherein the magnetic resonance control interface is adapted for controlling a magnetic resonance apparatus that is further adapted for performing magnetic resonance imaging thermometry, wherein the ultrasonic control interface is adapted for controlling an ultrasound apparatus adapted for applying ultrasonic therapy to a treatment zone (260), wherein the control module is further adapted for continuously monitoring the temperature of the subject in a region surrounding the treatment zone (262), wherein the control module is adapted for modifying the planning data in real time based upon the temperature in the region surrounding the treatment zone.

10. A therapeutic apparatus (100), the therapeutic apparatus comprising:
a control apparatus (106) according to claim 1,
a therapeutic ultrasound system (102),
a magnetic resonance apparatus (104).

11. A method for controlling a therapeutic apparatus (100), the method comprising:
acquiring magnetic resonance imaging data (400) with a magnetic resonance apparatus (104),
processing the magnetic resonance imaging data (402) with an image processing module (124, 126) to generate at least one magnetic resonance image (380, 500),
acquiring magnetic resonance spectroscopy data (404) with a magnetic resonance imaging apparatus (104),
processing the magnetic resonance spectroscopy data (406) with the image processing module (124, 128) to generate at least one magnetic resonance spectroscopy map (382, 502, 514, 516, 518, 520),
outputting the magnetic resonance imaging image and the magnetic resonance spectroscopy map (408) to a planning module (120),
receiving planning data (410) from the planning module,
controlling the treatment (412) of a subject (244) with a therapeutic ultrasound system using the planning data.

12. The method of claim 11, wherein the method further comprises the steps of:
displaying the magnetic resonance imaging image and the magnetic resonance spectroscopy map on a display (378) such that the magnetic resonance imaging image and the magnetic resonance spectroscopy map are superimposed,
receiving selection data from an operator using an editing interface, wherein the selection data indicates at least one region (624) of the displayed magnetic resonance image and the displayed magnetic spectroscopy map to be treated with ultrasonic therapy,
generating the planning data using the selection data.

13. The method of claim 11, wherein the method further comprises the steps of:
registering the location of the subject with the magnetic resonance image before acquiring the magnetic resonance spectroscopy data,
registering the location of the subject again before commencing the step of controlling the treatment of a subject with the therapeutic ultrasound system.

14. The method of claim 11, wherein the method further comprises the step of performing post evaluation treatment evaluation of the subject with magnetic resonance imaging.

15. A computer program product comprising machine executable code for performing method of claim 11 on a control apparatus for a therapeutic apparatus. A control apparatus and a method for controlling a therapeutic apparatus.
